(19) United States
(12) Patent Application Publication
(10) Pub. No.: US 2019/0339279 A1
(43) Pub. Date: Nov. 7, 2019

(54) DETECTING NITROAROMATIC COMPOUNDS WITH PYRENE-LABELED STARCH NANOPARTICLES

(71) Applicant: GreenMark Biomedical Inc., East Lansing, MI (US)

(72) Inventors: Jean DUHAMEL, Waterloo (CA); Lu LI, Waterloo (CA); Sanjay Rajnikant PATEL, Wasaga Beach (CA)

(21) Appl. No.: 16/401,651

(22) Filed: May 2, 2019

Related U.S. Application Data
(60) Provisional application No. 62/667,029, filed on May 4, 2018.

(51) Int. Cl. G01N 33/58

(52) U.S. Cl. CPC G01N 33/582

(57) ABSTRACT

Starch nanoparticles (SNPs) were fluorescently labeled with 1-pyrenebutyric acid and pyrene fluorescence was employed to detect nitrated organic compounds (NOCs) in solution and on paper surfaces. Fluorescence quenching of the pyrene-labeled SNPs (Py-SNPs) by NOCs such as nitromethane, nitrotoluene (MNT), dinitrotoluene (DNT), and trinitrotoluene (TNT) was characterized in DMSO and water. Since pyrene is insoluble in water, the fluorescence of the pyrene excimer that dominated the fluorescence spectrum of the Py-SNPs dispersed in water was used for the fluorescence quenching experiments. The efficient binding of the aromatic NOCs to the pyrene aggregates of Py-SNPs dispersed in water was used to detect NOCs by Py-SNPs adsorbed at the surface of paper sheets. The low quantities of aromatic NOCs detected by the Py-SNPs demonstrate the potential of Py-SNP-coated paper for the detection of such compounds.

### RELATED APPLICATIONS

This application claims the benefit of U.S. Provisional Application Ser. No. 62/667,029, filed May 4, 2018, which is incorporated herein in its entirety.

### FIELD

This application relates to pyrene-labeled starch nanoparticles and to the detection of nitroaromatic compounds

### BACKGROUND

Nitroaromatic compounds are well known for their utilization as explosives. They are widely used in the industry but unfortunately, they are also mutagenic to humans and toxic to the environment. Consequently, the ability to detect their presence is of the upmost importance. Current methods employed for nitroaromatic detection include the use of ion mobility spectroscopy, mass spectroscopy, or canine units. Although these methods offer high sensitivity, typically on the order of ppb and ppt, they suffer from high cost, non-portability, and complex instrumentation.

Currently, the most common technology for trace explosive detection, as seen in US airports, is ion mobility spectrometry (IMS). This method is similar to mass spectrometry and requires molecular ionization which makes the instrument bulky and expensive. In addition, since the amount of explosives is difficult to quantify using IMS, these instruments are usually used to raise an alarm rather than for quantification. Owing to the disadvantages of IMS, the suitability of alternate detection methods based on surface enhanced Raman spectroscopy, electronic olfactory systems, and sensor techniques has been investigated.

### INTRODUCTION

In compounds and methods described in this specification, modified starch nanoparticles (SNPs) are made and used to detect one or more chemicals of interest, for example a chemical in solution. SNPs are highly branched, offer an open architecture, and present a high surface area that leads to favorable interactions with other chemicals. Optionally, commercially available SNPs can be used as a starting material.

In some examples, the SNPs are fluorescently labeled, for example with pyrene. The labeled SNPs can be used in a sensor-based technique to detect chemicals such as nitroaromatic compounds. In some examples, pyrene-labeled starch nanoparticles (Py-SNPs) are used to detect minute quantities of nitrated aromatics such as trinitrotoluene, a known explosive. In some examples, the Py-SNPs are used to detect nitrated aromatics on a surface or in solution, optionally using a hand-held device. In some examples, the Py-SNPs provide a quantitative determination of the amount of explosive

Due to their high affinity to paper, Py-SNPs can be coated onto a sheet of paper, which can be used to wipe a surface to be tested for exposure to an explosive. The paper can be scanned, optionally in a hand held device, and the fluorescence of the Py-SNPs can be sued to assess whether the suspicious surface has been exposed to an explosive, such as a nitrated aromatic.

SNPs can be labelled with pyrene and their interior is sufficiently flexible to enable the formation of hydrophobic pyrene aggregates that provide the loci where hydrophibic molecules such as nitrated aromatics can bind. This phenomenon enhances the binding of hydrophobic molecules, which can then efficiently quench the fluorescence of pyrene. For example, once physically bound to the pyrene aggregates, nitrated aromatics quench the fluorescence of pyrene allowing the detection of the nitrated aromatics. Optionally using a hand-held device, a Py-SNP-coated tissue or other paper that has been used to swipe a suspicious surface can be probed to detect the presence of a pyrene fluorescence quenching substance such as a nitrated aromatic.

In other examples, fluorescently-labelled SNPs could also be used to detect the presence or, or measure the concentration of, heavy metals in water.

In examples described in this specification, the rate constants of quenching for several Py-SNPs are determined in DMSO and water in the presence of nitromethane, nitrotoluene and dinitrotoluene. Experimental data demonstrates that minute amounts of nitromethane, nitrotoluene of dinitrotoluene could quench the fluorescence of Py-SNPs, possibly due to the enhanced binding of the nitrated aromatics onto pyrene aggregates. Further examples demonstrate the quenching of Py-SNPs adsorbed onto filter paper by nitrotoluene and dinitrotoluene, which should also occur for nitromethane. While quenching has been observed for the Py-SNPs adsorbed onto paper, the heterogeneity of the paper used in the examples has made it difficult to determine the exact limit of dinitrolouene detection.

In this work, the fluorescence of pyrene-labeled starch nanoparticles (Py-SNPs) was applied to detect minute quantities of nitroaromatic compounds. Starch nanoparticles (SNPs) offer several advantages compared to traditionally used latex particles prepared from vinyl monomers, as they are derived from starch, a natural, abundant and low cost polymer. Pyrene was chosen as the fluorescent dye, owning to its photophysical properties such as high quantum yield, high molar extinction coefficient, and hydrophobicity. Upon labeling SNPs with pyrene, hydrophobic pyrene-rich microdomains are generated that emit as excimer. These hydrophobic microdomains can be exploited to drive sparingly water-soluble nitroaromatic compounds to them. Since most nitroaromatic compounds are quenchers of fluorescence, the hydrophobic microdomains generated by pyrene offer an inherent means for the detection of nitroaromatics by monitoring their sensitivity to fluorescence quenching.

Nitrated aromatics have a very low solubility in water but can bind to and quench pyrene and pyrene aggregates very efficiently. This specification describes a method to covalently attach 1-pyrenebutyric acid onto starch nanoparticles (SNPs). In water, the pyrene-labelled SNPs (Py-SNPs) generate pyrene aggregates onto which nitrated aromatics bind strongly. Upon binding, the fluorescence of pyrene is quenches and the extent of fluorescence quenching can be quantified to determine the concentration of nitrated aromatics in water. Since SNPs bind strongly onto paper, paper coated with Py-SNPs can be used to detect minute quantities of nitrated aromatics.

### BRIEF DESCRIPTION OF FIGURES

FIG. 1: Reaction scheme for the synthesis of pyrene labeled starch nanoparticles.

FIG. 2: Plot of k_{q} as a function of degree of substitution (DS) for NM (▴), MNT (▪), and DNT (●). Filled and hollowed symbols correspond to Py-SNPs and molecular pyrene, respectively.

FIG. 3: Plot of Kₛ as a function of the pyrene content of the Py-SNP for MNT (▪), DNT (▴), and TNT (●).

FIG. 4: Plot of (^{W}F₀/^{E}F)/(F₀/F) as a function of quencher mass per mm² for MNT (□), DNT (Δ), TNT (◯), and naphthalene (×).

FIG. 5: Types of Quenching

FIG. 6: Steady-State Fluorescence

FIG. 7: Steady-State Fluorescence (with quenching)

FIG. 8: Time resolved fluorescence yields the average time a fluorophore spends in its excited state after excitation.

FIG. 9: Quencher Absorption in DMSO

FIG. 10: Quenching by Nitromethane in DMSO

FIG. 11: Quenching by Nitromethane in DMSO

FIG. 12: Quenching by Nitrotoluene in DMSO

FIG. 13: Quenching by Nitrotoluene in DMSO

FIG. 14: Quenching in DMSO

FIG. 15: Quenching by Nitromethane in Water

FIG. 16: Quenching by Nitromethane in Water

FIG. 17: Quenching by Nitromethane in Water

FIG. 18: Quenching by Nitrotoluene in Water

FIG. 19: Quenching by Nitrotoluene in Water]

FIG. 20: Comparison of Kₛ

FIG. 21: Drop Method: Py-SNP-coated filter papers

FIG. 22: Quenching studies on filter paper

FIG. 23: Actual change in color of the Py-SNP coated filter paper with increasing quencher concentration

### DETAILED DESCRIPTION

Our research uses starch nanoparticles labeled with the dye pyrene to detect these nitrated compounds. Starch nanoparticles or SNPs can be produced by extrusion of starch. Starch is an abundant biopolymer constituted of linear amylose and highly branched amylopectin. Since these SNPs are obtained via an extrusion process, SNPs are a safe and cost-effective nanomaterial. Furthermore, SNPs can readily adsorb onto polar surfaces such as filter paper and glass which constitutes an advantage for their readily incorporation into paper- or glass-based films or sensors.

Pyrene-labeled SNPs (Py-SNPs) will be employed to detect nitrated compounds via fluorescence quenching. Pyrene is a hydrophobic compound with long fluorescence lifetime. Pyrene and its derivatives have a high quantum yield and large molar extinction coefficient, enabling us to work at low dye concentrations. Additionally, pyrene can form excimer, a complex formed upon the encounter between an excited and a ground-state pyrene. Since pyrene is hydrophobic, Py-SNPs in water generate hydrophobic microdomains where the high local pyrene concentration favors excimer formation. Hydrophobic quenchers, like many nitrated aromatic compounds such as mono-(MNT), di-(DNT), and tri-(TNT) nitrotoluene, will be driven to bind to these hydrophobic microdomains in water which will result in the fluorescence quenching of the excimer, since most nitroaromatic compounds are efficient fluorescence quenchers.

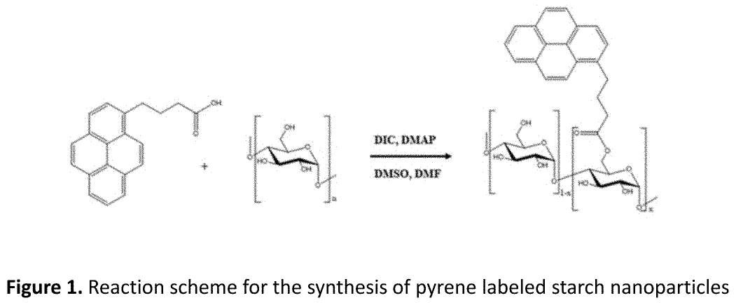

]The synthesis of Py-SNPs was done by a Steglich esterification as shown in FIG. 1. The SNPs were dispersed in a 3:1 dimethyl sulfoxide (DMSO):dimethyl formamide (DMF) mixture. 1-Pyrenebutyric acid and dimethylaminopyridine (DMAP) were added to the mixture, which was stirred for 30 minutes. The mixture was then put in an ice bath while diisopropylcarbodiimide (DIC) was added dropwise under continuous stirring. The mixture was covered in aluminum foil, to prevent photodegradation of the pyrenyl moieties by exposure to light, and was allowed to stir for 48 hours under nitrogen atmosphere. After 48 hours, the Py-SNPs were purified by precipitation in tetrahydrofuran (THF).

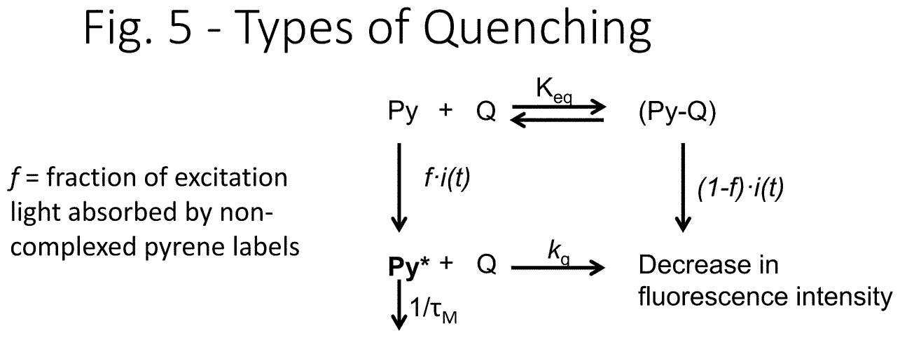

There are different types of quenching and some common experimental problems. The two main types of quenching encountered when conducting fluorescence quenching experiments are dynamic and static quenching. In dynamic quenching, an excited dye, in our case pyrene, will collide with a quencher. Upon contact with the quencher, the excited pyrene transfers its excess energy to the quencher which results in a decrease in the overall fluorescence intensity. The second type of quenching is static quenching, which is a process whereby a ground-state complex is formed between the quencher and the dye. Upon excitation of the dye, the dye is instantaneously quenched as it is complexed with the quencher and an overall decrease in fluorescence intensity will be observed. A mixture of both static and dynamic quenching can also occur. FIG. 5 shows types of quenching.

A common problem encountered in fluorescence quenching experiments is quencher absorption in a wavelength range where the dye absorbs or emits. In this situation, the light absorbed or emitted by the dye is absorbed by the quencher and the fluorescence intensity of the dye is substantially reduced. Luckily, quencher absorption only affects steady-state fluorescence measurements but not time-resolved fluorescence measurements. This distinction enables us to assess whether a decrease in the fluorescence intensity of a dye is the result of actual quenching by a quencher or mere reabsorption of light by a pseudo quencher.

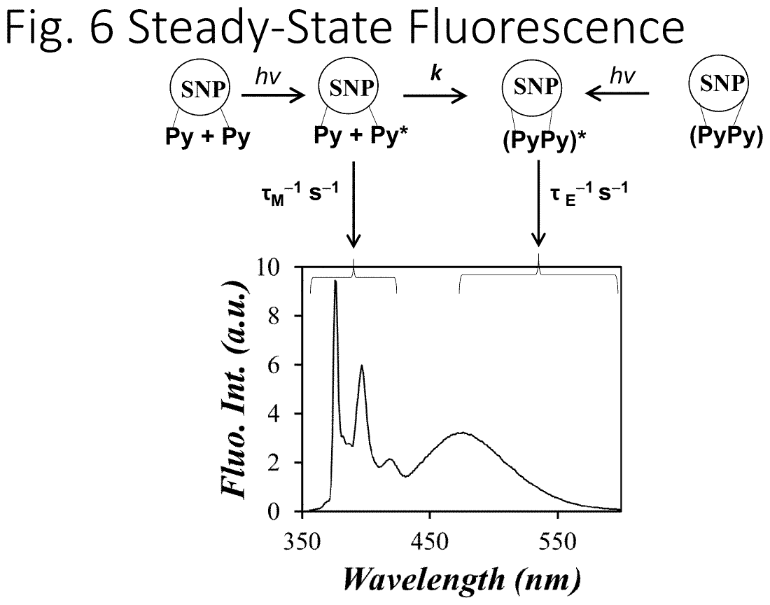

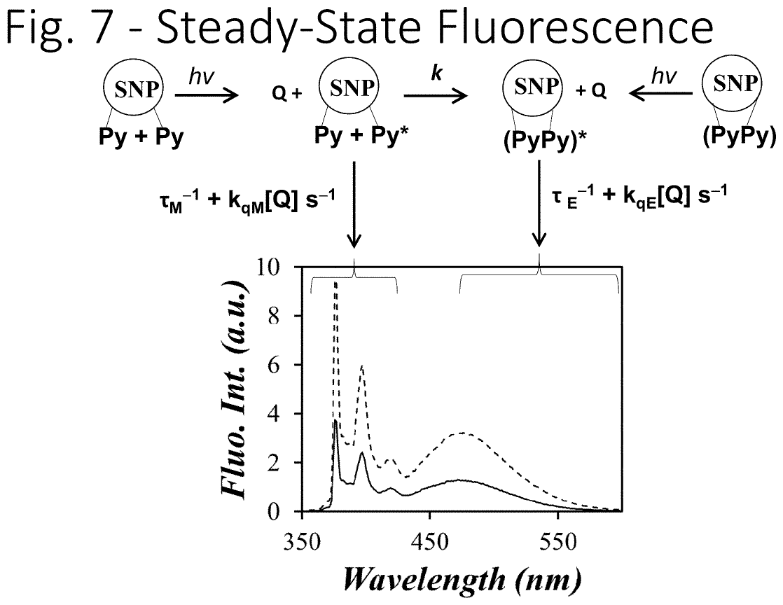

One of the instruments used in fluorescence quenching studies is a steady-state fluorometer, which is used to acquire the fluorescence spectra. In a steady-state fluorometer, the sample is continuously irradiated with light (λₑₓ=346 nm) which is absorbed by the dye (pyrene in our case). After excitation, pyrene can either emit as a monomer with peaks from about 360 to 400 nm or diffuse in solution and encounter another ground-state pyrene to form an excimer, whose broad structureless emission is centred around 480 nm. An excimer can also be formed by direct excitation of pre-associated pyrene aggregates. When a quencher is added to this solution, the overall spectrum intensity should decrease as shown in FIGS. 6 and 7.

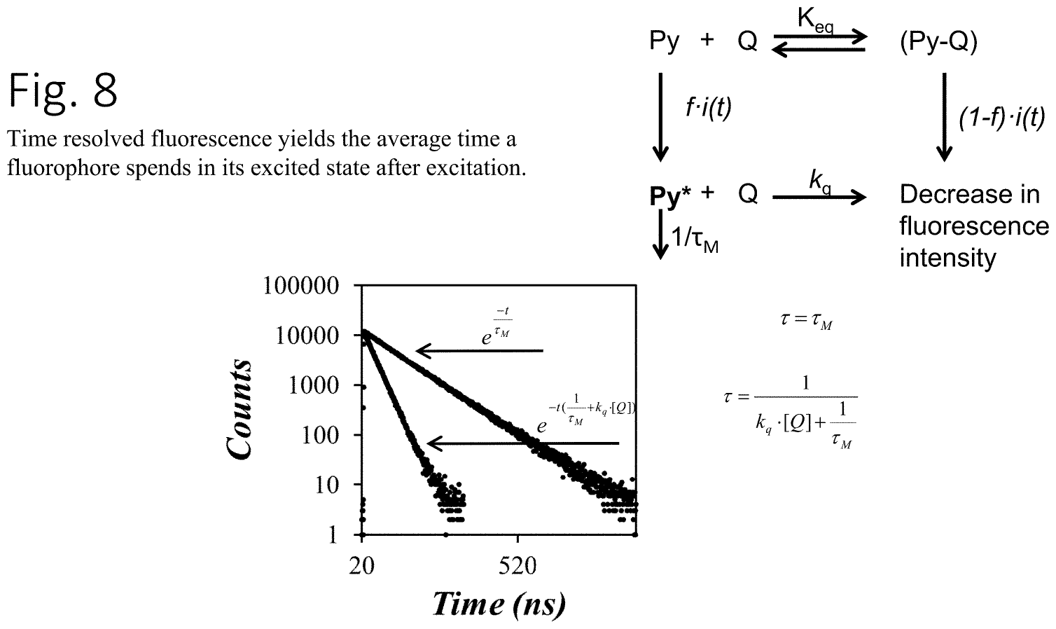

Another instrument used in fluorescence quenching studies is a time-resolved fluorometer. Time-resolved fluorescence measurements describe how quickly an excited fluorophore decays to its ground-state as shown in FIG. 8. In the absence of quencher, relaxation of the exited fluorophore to the ground-state proceeds through a single pathway and the exponential decay can be fitted with a single exponential [f(t)=exp(−t/τ_{M})]. Analysis of the fluorescence decay yields the natural lifetime Tm of the fluorophore. In the presence of a quencher, the average time that the dye spends in its excited state decreases, and therefore a decrease in τ is observed. A few things to point out here is that time-resolved fluorescence measurements can only be used to probe dynamic quenching, since in the case of static quenching, the fluorophore is instantaneously quenched. Similarly, if the excitation light or fluorescence are absorbed by the quencher, the fluorescence intensity of the fluorophore is reduced but the decay profile is unchanged. Thus time-resolved fluorescence decays can only probe photophysical processes that occur over time such as dynamic quenching, but cannot probe processes such as static quenching that involves the formation of a quencher-dye complex or absorption of the excitation or fluorescence of the dye which all occur instantaneously. See FIG. 8

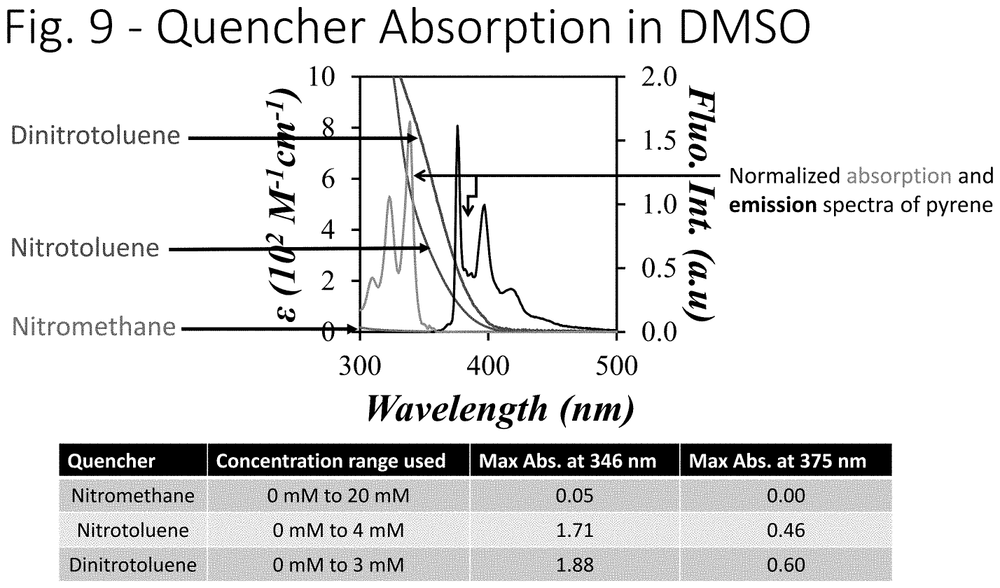

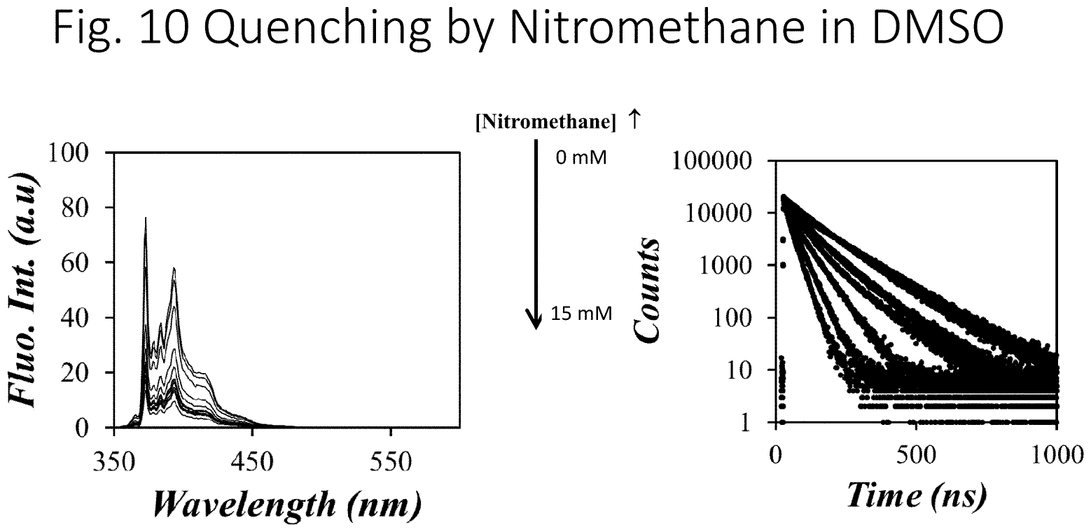

As mentioned before, a common problem encountered in fluorescence quenching measurements is quencher absorption. To assess the feasibility of such an eventuality, we determined the molar extinction coefficient of the quenchers used. The molar extinction coefficients of the quenchers and the normalized absorption and emission spectra of 1-pyrenebutyric acid were plotted on a same graph. At the highest concentration of quencher used, the absorption for nitromethane (NM) is very small, but that for 4-mononitrotoluene (MNT) and 2,6-dinitrotoluene (DNT) would equal 1.7 and 1.9, and 0.5 and 0.6 at the excitation (346 nm) and emission (376 nm) wavelength of the pyrene derivative, respectively. This high quencher absorption for MNT and DNT should be kept in consideration when conducting the quenching studies since they will affect the fluorescence intensity of the Py-SNPs. See FIG. 9.

The first quenching study was conducted with nitromethane in DMSO. As expected, a decrease in the fluorescence intensity of the spectra can be seen with increasing quencher concentration, and the fluorescence lifetime decreased as well. See FIG. 10.

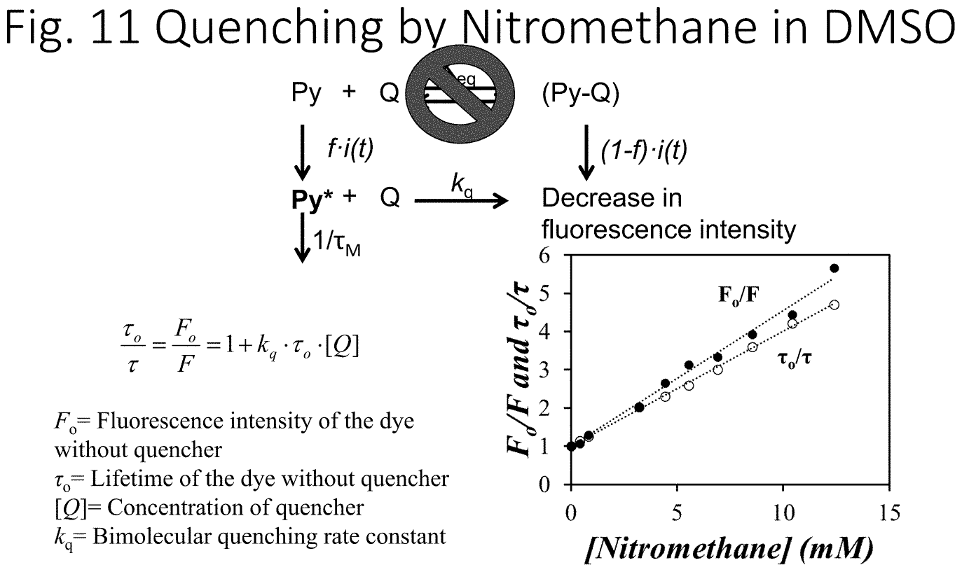

Typically, Stern-Volmer plots are used to determine the bimolecular quenching rate constant k_{q}. This parameter can be obtained from a Stern Volmer plot, namely a plot of the ratios F₀/F or τ₀/τ as a function of quencher concentration. In the absence of static quenching, both the F₀/F and τ₀/τ ratio should increase linearly with quencher concentration. From our experimental results, we find good agreement between the F₀/F and τ₀/τ ratios, and the slight discrepancy observed is most likely due to residual static quenching. It should be noted that the slopes from the τ₀/τ ratios, which are unaffected by static quenching or re-absorption, were used to determine all k_{q} values. See FIG. 11.

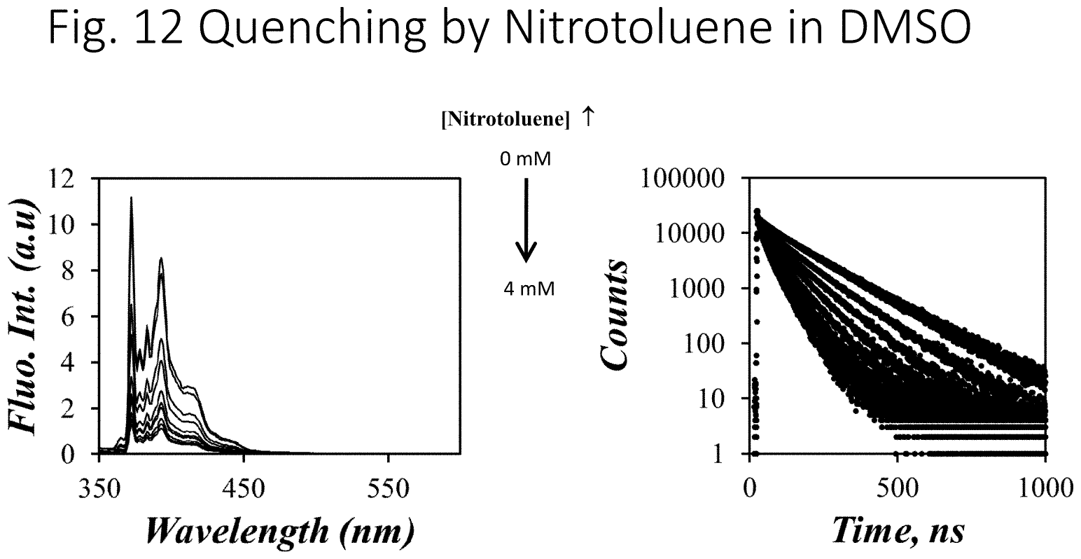

The quenching study was repeated with MNT. As observed before, a decrease in intensity and lifetime was observed with increasing quencher concentration in both the steady-state fluorescence spectra and the time-resolved fluorescence decays, respectively. See FIG. 12.

However, upon examination of the Stern-Volmer plots, an exponential increase in F₀/F was observed while τ₀/τ increased linearly with increasing quencher concentration. At first glance this might indicate a mix of static and dynamic quenching.

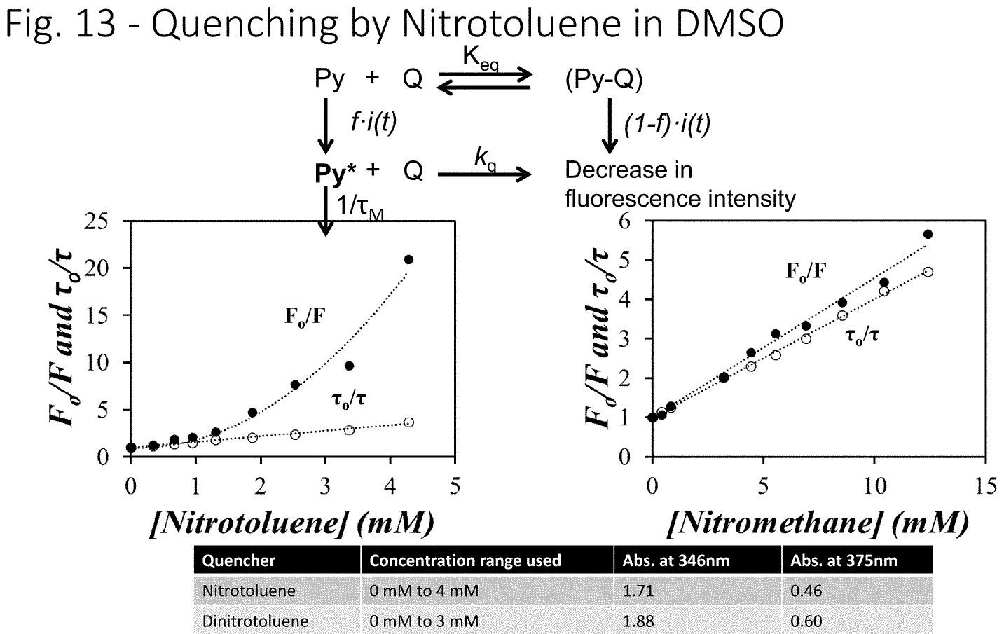

However keeping in mind that the absorption of a 4 mM concentration of MNT solution would equal 1.7 and 0.5 at the excitation and emission wavelengths, respectively, the light absorbed and emitted by pyrene is most certainly being absorbed by MNT. Thus, the exponential increase in F₀/F is most likely due to quencher absorption and not static quenching. The same trends shown here for MNT was also observed for DNT. On the other hand, the linear increase observed for τ₀/τ describes the dynamic quenching of pyrene by MNT and the slope yields the k_{q} value for the quenching of pyrene by MNT. See FIG. 13.

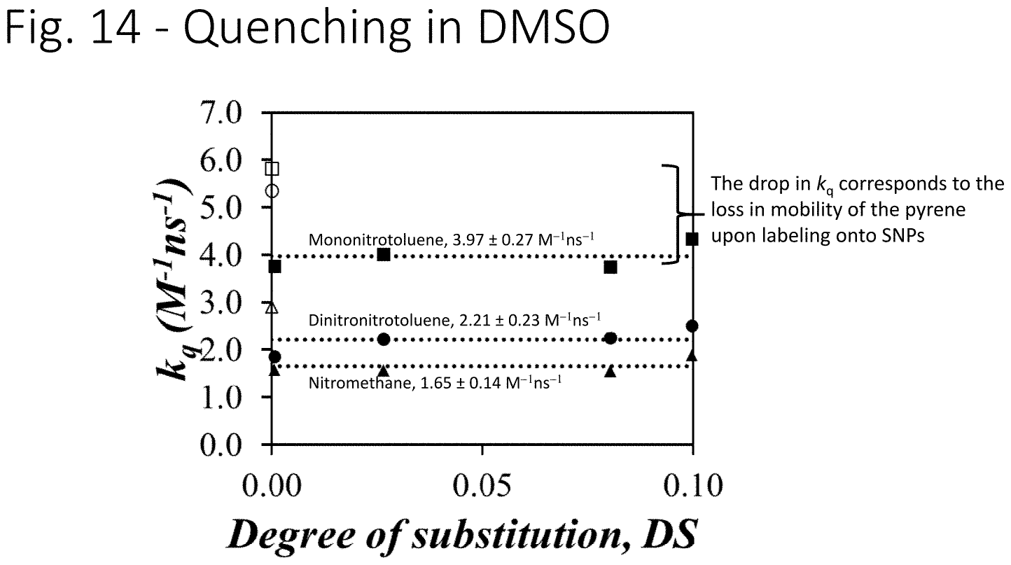

FIG. 14 is a plot of the k_{q} values obtained for all samples in DMSO as a function of the degree of substitution (DS) in 1-pyrenebutyric acid attached onto the SNPs. The hollow symbols represent quenching studies conducted with molecular pyrene, while the filled symbols represent quenching studies conducted with Py-SNPs. The drop in k_{q} between molecular pyrene and Py-SNPs for each quencher was attributed to the loss in mobility experienced by the pyrene label when attached onto the SNPs. For all Py-SNPs, k_{q} remained constant with DS, within experimental error, suggesting that in DMSO, all the pyrene labels were equally accessible to the quenchers. Furthermore, k_{q} for MNT seemed to be higher compared to that for DNT and NM. A combination of hydrophobicity of the nitroaromatic compounds and H-bonding with the starch hydroxyls provides a rational for this trend.

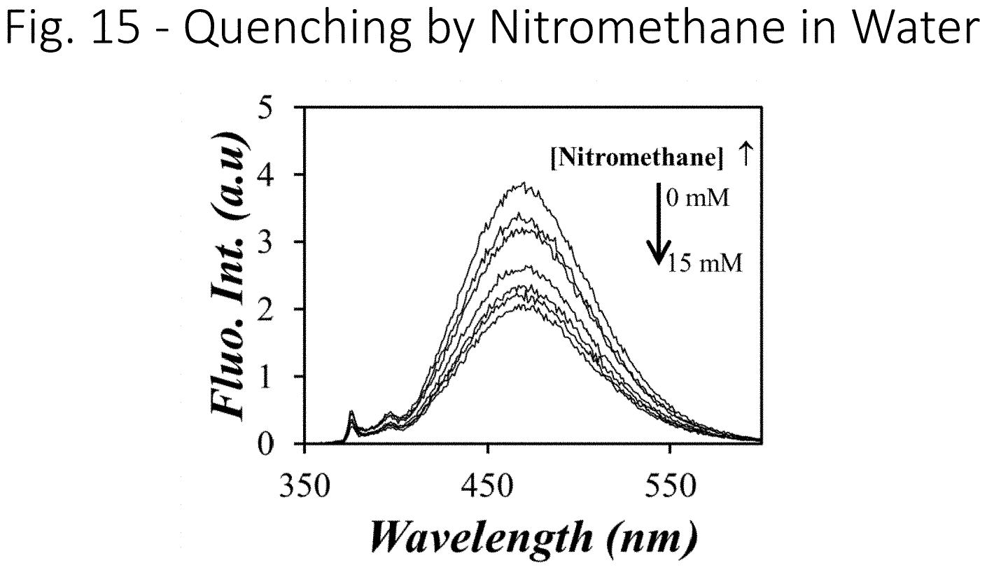

The next set of quenching studies were conducted in water. Nitromethane, a water-soluble quencher, is not expected to target the hydrophobic microdomains generated by the pyrene aggregates of the Py-SNPs in water. As seen in the steady-state fluorescence spectra, a decrease in the overall fluorescence intensity in the spectra was observed with increasing NM concentration. See FIG. 15.

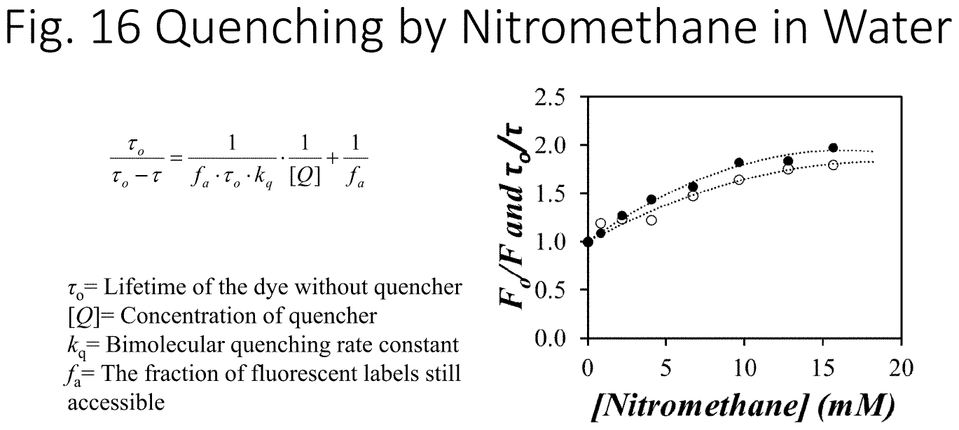

A downwards curvature was observed in the Stern-Volmer plots. This is typically due to protective quenching, and can be handled by a modified Stern-Volmer equation/plot. Using this modified Stern-Volmer plot, we were able to obtain k_{q} and fₐ, which is the fraction of pyrene labels accessible to the quencher. See FIG. 16.

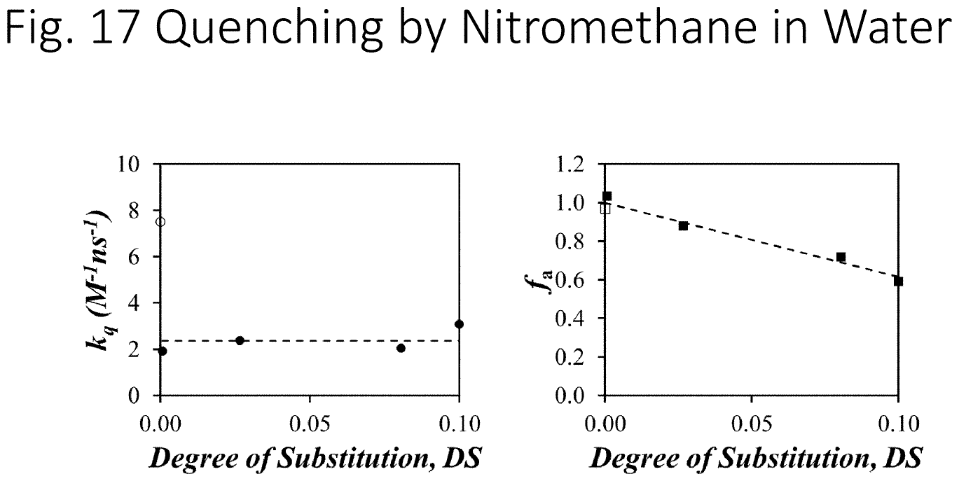

As previously observed for DMSO, k_{q} remained constant regardless of pyrene content once we accounted for the fraction of inaccessible pyrene labels. We also observed that fₐ decreased with increasing pyrene content. A more hydrophobic Py-SNP seemed to shield isolated pyrene monomers from quenching by NM. See FIG. 17.

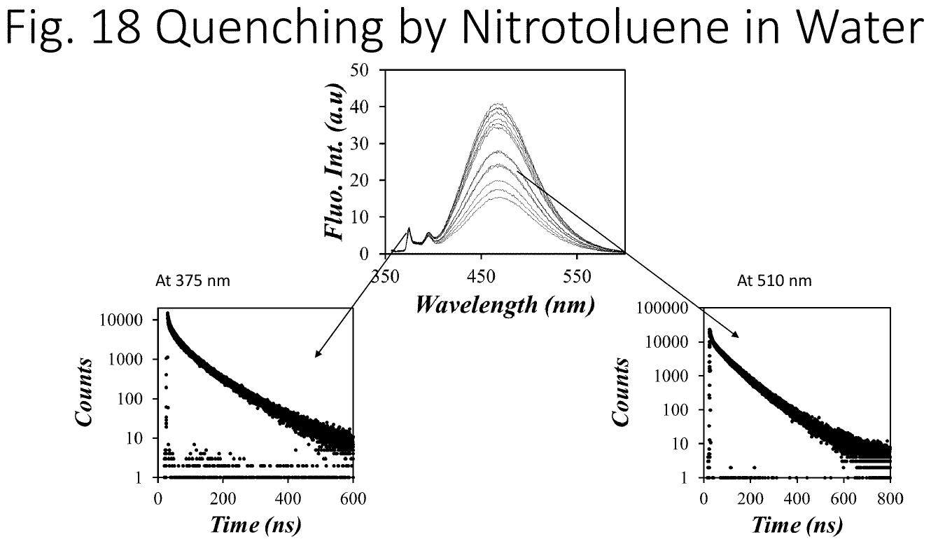

The next study conducted was with the Py-SNPs quenched by MNT in water. Interestingly, as we progressively increased the quencher concentration, a substantial decrease was observed in the excimer fluorescence intensity which was accompanied by only a 10% decrease in the fluorescence intensity of the monomer. Furthermore, when we compared the fluorescence decays of both the monomer (346 nm) and excimer (510 nm), little change was observed. Combining the substantial decrease in the excimer fluorescence intensity with the absence of change in the monomer and excimer decays, we concluded that the mechanism for the quenching of the pyrene excimer by MNT was mainly static in nature and, more importantly, the hydrophobic quencher, MNT, seemed to specifically target the hydrophobic microdomains generated by the pyrene aggregates. Similar trends in the fluorescence spectra and decays were observed for all the Py-SNP samples when quenched by DNT or TNT. See FIG. 18.

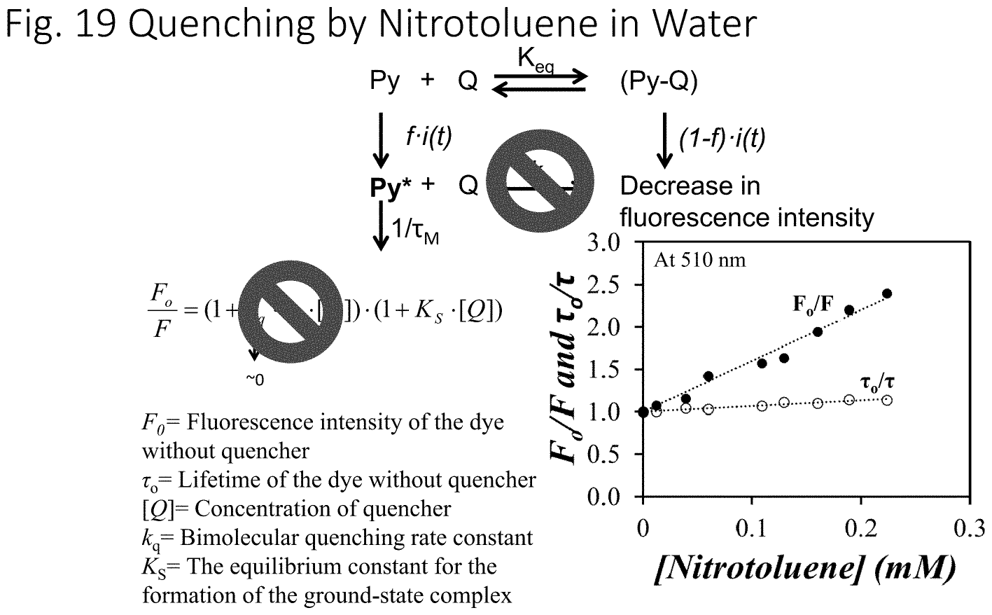

Since little change was observed in the fluorescence decays, the data could be treated as if only static quenching occurred. Furthermore, since little change was observed in the monomer peak (375 nm), the excimer fluorescence intensity (from 500 to 530 nm) was used to generate the Stern-Volmer plots and obtain Kₛ, the equilibrium constant for the formation of the ground-state complexes between the pyrene aggregates and the nitroaromatic compounds. One thing that should be noted here, is that quencher absorption should not be an issue since the quencher concentration used in water was much lower compared to the quencher concentration used in DMSO, due to the low solubility of the nitroaromatic compounds in water. This same analysis was applied to the quenching studies with DNT and TNT. See FIG. 19.

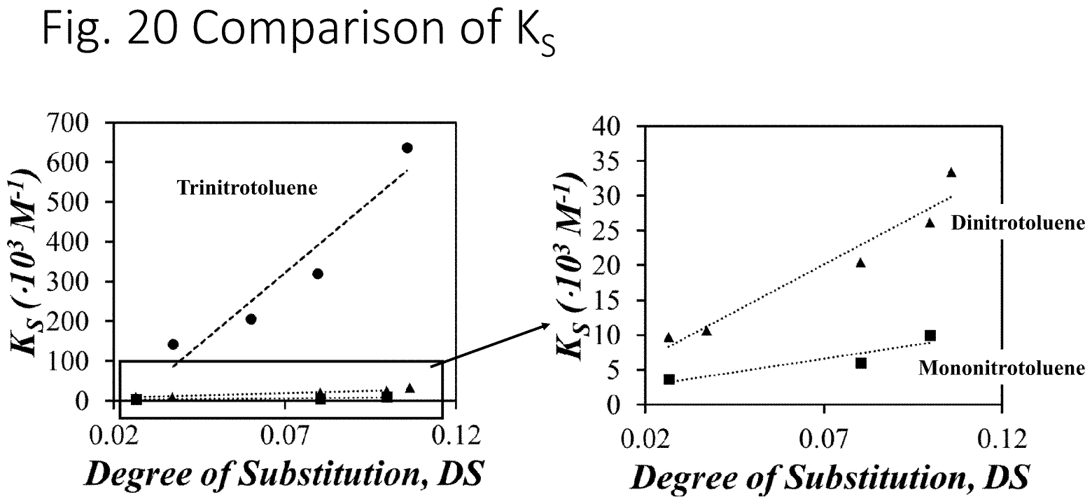

FIG. 20 includes a plot of Kₛ as a function of DS. Kₛ is seen to increase with increasing pyrene content, which is certainly due to the formation of hydrophobic microdomains of pyrene aggregates that are either larger in size or number. Additionally, it seems that TNT is a significantly better quencher of the excimer fluorescence for Py-SNPs in water compared to DNT and MNT. This may be a result of the extra nitro-group which seems to be responsible for the much increased Kₛ value.

Up until this point, we have shown that quenching of Py-SNPs in DMSO and water occurred by dynamic and static quenching, respectively. The focus of the remaining talk will be the use of these Py-SNPs to make sensor strips with filter paper that can be used for detection purposes.

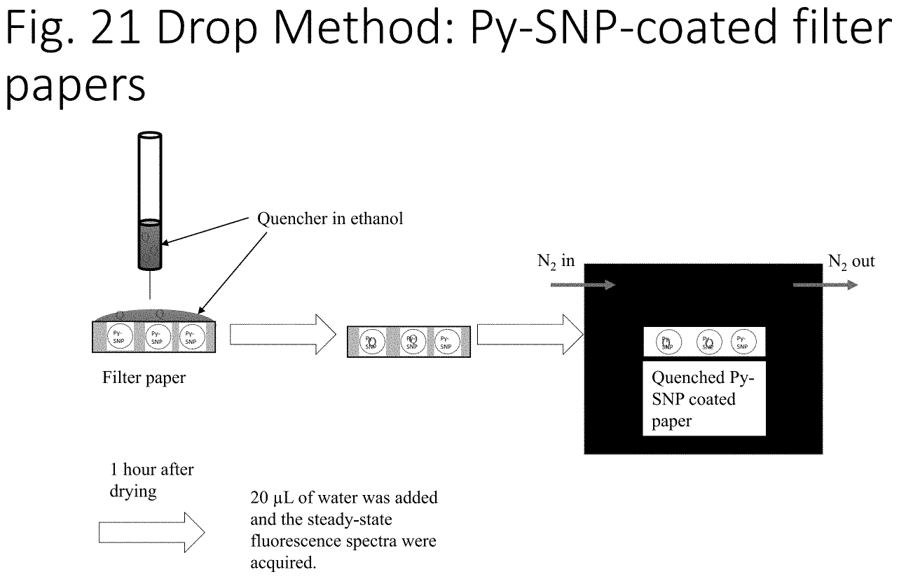

The method that was chosen to coat the filter paper was the drop method. This method uses an aqueous dispersion of Py-SNPs which is directly deposited onto a piece of filter paper of known size. This filter paper was then dried under N₂ gas in the dark. Once the filter paper was completely dried, 20 μL of water was added to the Py-SNP-coated filter paper and the fluorescence spectrum was acquired. This provides F₀ (the fluorescence intensity without quencher). A known amount of quencher solution in an organic solvent (ethanol) was deposited onto the Py-SNP-coated filter paper. The ethanol was evaporated with a slow stream of N₂ in the dark. Once completely dry, 20 μL of water was added onto the filter paper again and the fluorescence intensity was acquired (F). This method was used for all filter paper based quenching studies. See FIG. 21.

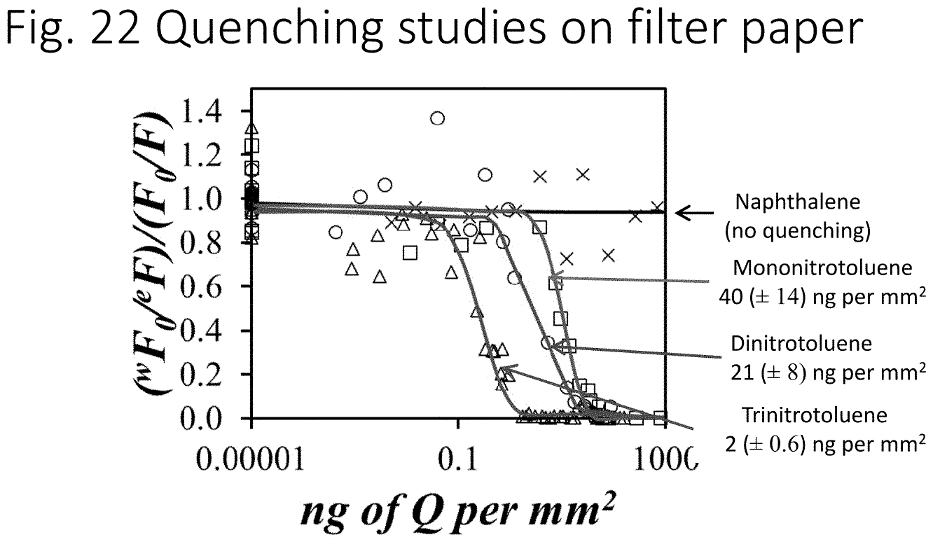

Quenching studies on the Py-SNP-coated filter papers were conducted with MNT, DNT, TNT and naphthalene, this latter compound being expected not to quench the fluorescence of pyrene. When ethanol was applied to the Py-SNP-coated filter papers, a change in the fluorescence intensity was observed even without quencher. To account for this change, the F₀/F ratios were all normalized to the change (^{w}F₀/^{e}F₀) in the fluorescence intensity observed when the filter papers without quencher were impregnated with water (w) or ethanol (e). All the detection limits reported here correspond to the quencher concentration where 100% quenching occurred. From these quenching studies, similar trends to those observed in water were found. TNT was a substantially better quencher compared to DNT and MNT. Furthermore, using naphthalene as a representative aromatic quencher, we found that at even high concentrations of this aromatic compound, little change in the fluorescence intensity of the Py-SNPs was observed. See FIG. 22.

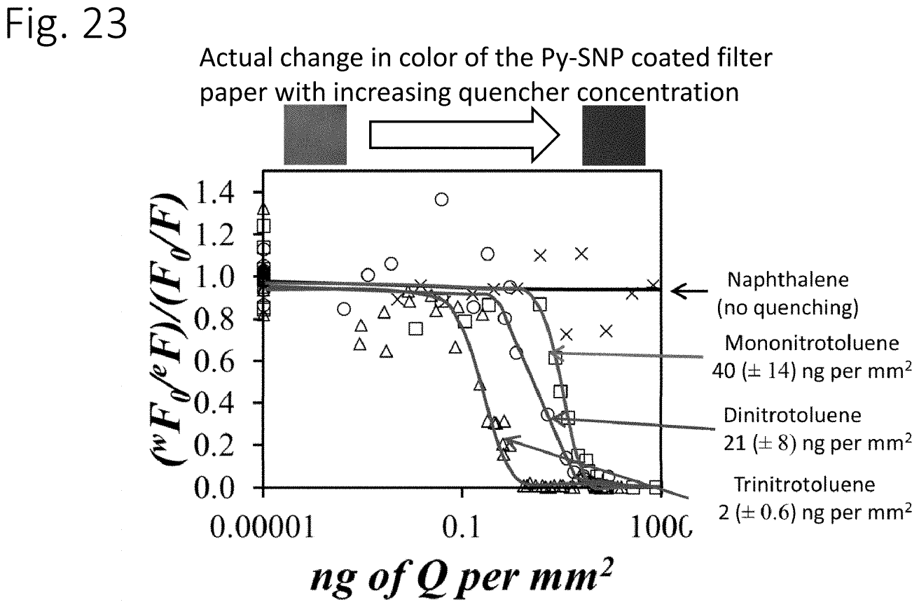

Not only did we characterize the quenching of Py-SNPs in solution (DMSO and water) by several nitroaromatic compounds, but we were also able to demonstrate the potential use of Py-SNP-coated filter papers as a sensor for nitroaromatic compounds. Furthermore, the detection limit we report here corresponds to the quencher concentration where 100% quenching occurs and can be easily observed by the naked eye under a black light, as we go from a blue filter paper to essentially a colourless one. See FIG. 23.

Other common explosive compounds or contaminants such as picric acid might also be detected. Probes other than pyrene, such as naphthalene, might also be used for detection applications.

### Examples

Pyrene-Labeled Starch Nanoparticles (Py-SNPs) Synthesis: The Py-SNP samples used in this research were synthesized according to the reaction scheme shown in FIG. 1. The synthesis and purification of the Py-SNPs have been described in Yi, W. Characterization of Starch Nanoparticles by Fluorescence Techniques, M.Sc Thesis, University of Waterloo, 2014, which is incorporated herein by reference in its entirety.

Steady-State Fluorescence: All steady-state fluorescence spectra were acquired on a Photon Technology International LS-100 fluorimeter equipped with a Xenon Arc lamp. All samples were excited at 346 nm and the emission spectra were acquired from 356 to 600 nm. The fluorescence intensities for the monomer (Fₘ) and excimer (Fₑ) were calculated by integrating the fluorescence signal from 372 to 378 nm and 500 to 530 nm, respectively. All quenching studies conducted on Py-SNP-coated filter paper were carried out using front face geometry. All fluorescence spectra acquired for the Py-SNP-coated filter paper was background corrected with unlabeled SNP-coated filter paper.

Time-Resolved Fluorescence: All time-resolved fluorescence decays were acquired on an IBH fluorimeter equipped with an IBH 340 nm NanoLED. All solutions were excited at 346 nm and the fluorescence decays for the Py-SNPs were acquired at 375 and 510 nm for the monomer and excimer, respectively. To ensure a good signal-to-noise ratio, the fluorescence decays were acquired with 20,000 counts at the decay maximum. All decays were fitted with a sum of exponentials. For all the decay fits, a χ² value between 0.98 and 1.20 was obtained with the residuals and autocorrelation function of the residuals randomly distributed around zero, thus demonstrating a good fit.

Quenching studies in solution: All quenching experiments conducted in DMSO were carried out at a pyrene concentration of 2.5 10⁻⁶ M, while progressively increasing the concentration of quencher. The selected pyrene concentration, corresponding to an absorbance of 0.1 at 346 nm, ensured minimal particle-particle interactions. A stock solution of Py-SNPs ([Py]=3.4·10⁻⁶ M) was made in DMSO. The stock solution (3.7 g) was diluted with 1.3 g of DMSO to yield the solution “Sol A” with a pyrene concentration of 2.5·10⁻⁶ M, corresponding to an absorbance of 0.1. Stock solutions of the quenchers, namely nitromethane (NM, 0.2 M), 4-nitrotoluene (MNT, 0.04 M) and 2,6-ditrotoluene (DNT, 0.04 M) were made in DMSO. The stock solutions with quencher (1.3 g) were diluted with 3.7 g of the Py-SNP stock solution in DMSO, yielding the solution “Sol Q” with a same pyrene concentration as Sol A. The fluorescence spectrum and decay at 375 nm were acquired for Sol A to determine the fluorescence intensity (F₀) and lifetime (τ₀) of the pyrene monomer without quencher. Then known quantities of Sol Q was added to the cuvette directly and the fluorescence intensity (F) and decay lifetime (τ) of the pyrene monomer with quencher were determined. This process was repeated until 10 data points were obtained. Since Sol A and Sol Q had the same concentration of Py-SNPs, this procedure enabled to progressively increase the quencher concentration while maintaining the same Py-SNP concentration. Quenching studies conducted in water were conducted in a similar manner as in DMSO. A Py-SNP stock solution was prepared in DMSO (4.6·10⁻⁴ M), and 0.06 g of this stock solution was diluted with 8 g of milliQ water to yield an aqueous solution of Py-SNP with a pyrene concentration of 3.4·10⁻⁶ M. This water stock was subsequently used to prepare 5 g of Sol A and Sol Q, using water to dilute the samples. The final pyrene concentrations of the solutions, namely Sol A and Sol Q, was 2.5·10⁻⁶ M. All solutions were prepared in water with 0.8 wt % of DMSO.

Py-SNP-Coated filter papers: The drop method was developed to coat pieces of Whatman No1 filter papers with Py-SNP. A dispersion of Py-SNP in milliQ water was prepared with a final pyrene concentration of 3.2·10⁻⁵ M with 0.67 wt % DMSO. This stock solution (0.03 g) was deposited directly onto 1 cm×1 cm pieces of Whatman No1 filter paper, resulting in Py-SNP-coated filter paper with approximately 1.6·10⁻¹¹ mol of pyrene per mm² of filter paper. The resulting papers were dried under N₂ in the dark. A series of quenching solution using MNT, DNT, and TNT were prepared in ethanol or acetonitrile. A same volume of 10 μL of the different quenching solutions was deposited directly on the filter paper which was allowed to completely dry. The filter papers were rewetted with 10 μL of water and the fluorescence spectra were acquired. To account for the change in the fluorescence intensity due to the addition of ethanol when depositing the quencher solution, 4 pieces of paper were wetted with 10 μL of ethanol, allowed to dry, and rewetted with water. The ^{W}F₀/^{E}F₀ values were averaged among the 4 pieces of paper and plots of (^{W}F₀/^{E}F₀)/(F₀/F) as a function of quencher mass per mm², where ^{W}F₀ and ^{E}F₀ are the fluorescence intensities of Py-SNP-coated filter papers with no quencher before and after ethanol addition, respectively. F₀ and F are the fluorescence intensity of the filter paper without and with quencher, respectively.

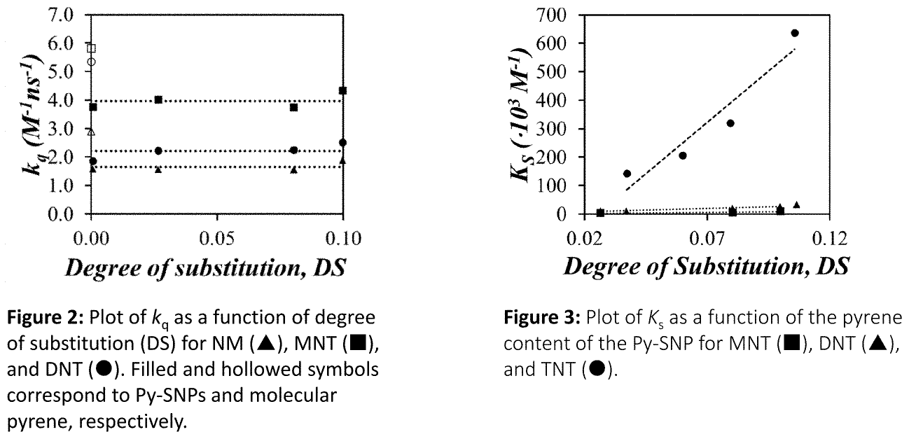

Quenching studies in DMSO: Quenching studies were conducted with Py-SNP dispersions in DMSO as nitromethane (NM), nitrotoluene (MNT), dinitrotoluene (DNT), and the pyrene labels are soluble and SNPs are dispersible in DMSO. From the steady-state fluorescence (SSF) spectra and time-resolved fluorescence (TRF) decays, Stern-Volmer plots of F₀/F and τ₀/τ were constructed and the bimolecular quenching rate constants were determined using the τ₀/τ ratios. As expected, F₀/F and τ₀/τ increased linearly with increasing NM concentration. A good overlap between the trends obtained with F₀/F and τ₀/τ was indicative of dynamic quenching being the predominant mode of quenching. Quenching studies conducted with MNT and DNT showed a linear and exponential increase of, respectively, the τ₀/τ and F₀/F ratios with increasing quencher concentration. Typically, the combination of an exponential increase for F₀/F and linear increase for τ₀/τ is indicative of mixed dynamic and static quenching. However at concentrations of 4 and 3 mM for MNT and DNT, the absorption of the dispersion would equal 1.7 and 1.9 at 346 nm, respectively. Such absorbances are too high for fluorescence measurements because they hinder access of the excitation beam to the center of the cell, which decreases the fluorescence intensity resulting in the exponential increase in the F₀/F ratio. Fortunately excessive absorption does not affect the TRF measurements, implying that the bimolecular quenching rate constant k_{q} obtained from the slope of τ₀/τ represented as a function of quencher concentration were reliable. Upon plotting the k_{q} values in FIG. 2, k_{q} was found to be independent of pyrene content. This result demonstrates that all pyrene labels were equally accessible to the quenchers in DMSO, as would be expected. k_{q} values of 1.7 (±0.1) M⁻¹ ns⁻¹, 4.0 (±0.3) M⁻¹ ns⁻¹, and 2.2 (±0.2) M⁻¹ ns⁻¹ were found for NM, MNT, and DNT, respectively. The efficiency of quenching, as reflected by the k_{q} values, was found to decrease as MNT>DNT>NM, where MNT and NM were the best and worst quencher, respectively. Interestingly, DNT was 1.8-fold less efficient compared to MNT. Since DMT has an extra nitro-group compared to MNT, DNT was expected to have a higher k_{q} value than MNT. The decrease in k_{q} for DNT compared to MNT was attributed to enhanced H-bonding between DNT and the starch hydroxyls which restricted the diffusion of DNT, thus restricting its mobility as it interacted with starch to quench the pyrene labels.

Quenching Studies in Water. Quenching studies, similar to those carried out in DMSO, were conducted in water. NM has a high solubility in water (10 g/L), whereas MNT, DNT, and TNT have a much lower water solubility (0.361 g/L, 0.279 g/L, and 0.127 g/L, respectively). Stern-Volmer plots obtained for the quenching with NM with Py-SNP samples with a degree of substitution (DS) of 0.0265 (2.65 mol % of pyrene labels per anhydroglucose unit) and lower followed similar trends a those observed in DMSO. However Py-SNP samples with a DS of 0.08 and higher resulted in Stern-Volmer plots with a downwards curvature. A downwards curvature in a Stern-Volmer plot is indicative of protective quenching. A modified Stern-Volmer equation was used to determine k_{q} and fₐ, the fraction of dyes accessible to the quencher. As observed before in DMSO, k_{q} remained constant in water regardless of the pyrene content when quenched by nitromethane. Furthermore, fₐ decreased linearly with increasing content of pyrene labels attached to the Py-SNPs. A decrease in fₐ suggests that, as more hydrophobic pyrene is attached to the Py-SNPs, the hydrophobic domains are less accessible to the water-soluble NM quencher.

Quenching studies were repeated in water for the Py-SNP samples with MNT, DNT and 2,4,6-trinitrotoluene (TNT). Addition of MNT, DNT, and TNT resulted in little change (<10%) in the fluorescence intensity of the pyrene monomer between 356 and 400 nm, but led to a substantial decrease (up to 60%) of the excimer fluorescence intensity between 430 to 600 nm. This result suggested that MNT, DNT, and TNT targeted the hydrophobic microdomains generated by the pyrene labels on the SNPs. The TRF decays acquired for the monomer at 375 nm and the excimer at 510 nm with increasing quencher concentration overlapped, demonstrating the absence of dynamic quenching. Together, the SSF and TRF results led to the conclusion that MNT, DNT, and TNT would target the hydrophobic domains on the Py-SNPs generated by the pyrene labels with a binding constant Kₛ. Quenching of pyrene excimer would happen instantaneously in a static manner for the quenchers bound to the pyrene aggregates. Considering the excimer fluorescence, a linear relationship was obtained between the F₀/F ratio and the quencher concentration whose slope yielded Kₛ. As shown in FIG. 3, Kₛ increased with increasing pyrene content. Increasing the pyrene content generated more hydrophobic microdomains, thereby resulting in increased binding of the hydrophobic quenchers. Kₛ for the different quenchers decreased according to the following sequence: TNT>DNT>M NT. The trend obtained with Kₛ implied that each additional nitro-group on the aromatic rings led to stronger binding of the quencher to the hydrophobic microdomains. Increasing the pyrene content generated more hydrophobic microdomains that led to stronger binding as indicated by an increase in Kₛ.

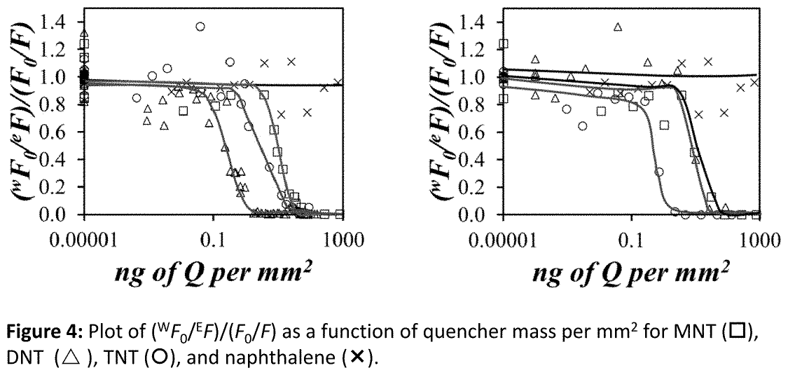

Py-SNP-Coated Papers: The use of Py-SNPs deposited onto a solid substrate was also investigated to develop a paper-based sensor. The Py-SNPs were deposited according to the drop method which was developed to coat filter paper with Py-SNPs and quenching studies were conducted on Whatman Filter Paper No1 with MNT, DNT, TNT, and naphthalene using a Py-SNP sample with a DS of 0.11. Detection limits of 80 (±10), 35 (±2), and 5 (±1) ng per mm² for MNT, DNT, and TNT, respectively, were determined in FIG. 4. Interestingly, the detection limit of TNT was about 10 and 16 fold lower compared to that of DNT and MNT, respectively. This decrease in the detection limit was also reflected by the Kₛ trends, where Kₛ for TNT was significantly higher compared to MNT and DNT. To demonstrate the selectivity of Py-SNP-coated filter papers, quenching studies were repeated with naphthalene as an aromatic contaminant. As seen in FIG. 4, no significant quenching was observed within experimental error, confirming that the quenching observed for the Py-SNPs was selective towards nitroaromatic compounds.

This study has demonstrated that the fluorescence of Py-SNP can be employed to detect minute quantities of nitroaromatic compounds via fluorescence quenching. Detection limits for Py-SNP in water where 50% quenching occurred were found to equal 1.1·10⁻⁴ M, 2.5·10⁻⁵ M, and 1.6·10⁻⁶ M for MNT, DNT, and TNT, respectively. The use of Py-SNP-coated filter papers was investigated. Detection limits for MNT, DNT, and TNT where 100% quenching occurred, was found to be 40 (±14), 21 (±8) and 2 (±0.6) ng per mm², respectively. Quenching studies with naphthalene, as an aromatic contaminant, demonstrated the selectivity of the Py-SNP-coated filter papers towards nitroaromatic compounds.

Starch nanoparticles (SNPs) were fluorescently labeled with 1-pyrenebutyric acid and pyrene fluorescence was employed to detect nitrated organic compounds (NOCs) in solution and on paper surfaces. SNPs were generated that contained 6-30 mol % 1-pyrenebutyric acid. Fluorescence quenching of the pyrene-labeled SNPs (Py-SNPs) by nitromethane, nitrotoluene (MNT), dinitrotoluene (DNT), and trinitrotoluene (TNT) was characterized in DMSO and water. Since pyrene is insoluble in water, the fluorescence of the pyrene excimer that dominated the fluorescence spectrum of the Py-SNPs dispersed in water was used for the fluorescence quenching experiments. Since pyrene and the aromatic NOCs are soluble in DMSO but not in water, quenching of pyrene by MNT, DNT, and TNT occurred in a dynamic and static manner in DMSO and water, respectively. By contrast, nitromethane being soluble in water and DMSO, quenching of Py-SNP took place in a dynamic manner in both solvents. Static quenching of pyrene by the aromatic NOCs in water took place at much lower quencher concentration in water than in DMSO due to the large binding constant of these quenchers to pyrene aggregates formed in the Py-SNPs dispersed in water. The efficient binding of the aromatic NOCs to the pyrene aggregates of Py-SNPs dispersed in water was taken advantage of to determine how little NOCs could be detected by Py-SNPs adsorbed at the surface of paper sheets. It was found that paper sheets coated with Py-SNPs could detect as little as 5 and 50 ng/mm² TNT and DNT, respectively. The low quantities of aromatic NOCs detected by the Py-SNPs demonstrate the potential of Py-SNP-coated paper for the detection of such compounds.

Since SNPs bind strongly onto paper, paper coated with Py-SNPs coated onto a substrate, for example paper can be used in a method of detecting nitrated aromatics. The method includes contacting a surface or solution to be tested with Py-SNPs and observing fluorescence of the Py-SNPs. A detection method may include wiping a surface to be tested with Py-SNPs coated onto a substrate, for example paper.

We claim:

1. A composition for detecting nitrated aromatic compounds comprising, a substrate; and
pyrene-labeled starch nanoparticles attached to the substrate.

2. The composition of claim 1 wherein the substrate comprises paper.

3. The composition of claim 2 wherein the nanoparticles are coated on a surface of the paper.

4. A method of detecting nitrated aromatic compounds comprising the steps of contacting a surface or solution to be tested with pyrene-labeled starch nanoparticles and observing fluorescence of the nanoparticles.

5. The method of claim 4 comprising wiping a surface to be tested with a substrate comprising the nanoparticles.

6. The method of claim 5 wherein the substrate comprises paper and the nanoparticles are coated on a surface of the paper.
